(19) United States
(12) Patent Application Publication
(10) Pub. No.: US 2017/0079843 A1
(43) Pub. Date: Mar. 23, 2017

(54) SYSTEM AND METHOD FOR REMOVING CORNEAL EPITHELIUM

(71) Applicant: STRATHSPEY CROWN HOLDINGS, LLC, Newport Beach, CA (US)

(72) Inventors: Guy M. Kezirian, Scottsdale, AZ (US); David Tat-Chi Lin, Vancouver (CA); David Haydn Mordaunt, Los Gatos, CA (US)

(21) Appl. No.: 14/861,811

(22) Filed: Sep. 22, 2015

(51) Int. Cl. A61F 9/008

(52) U.S. Cl. CPC A61F 9/00814; A61F 2009/00851; A61F 2009/00872; A61F 2009/00897

(57) ABSTRACT

In accordance with the present invention, a system and method are provided for removing the corneal epithelium from a patient's eye while monitoring the autofluorescent response that results during laser photoablation of the epithelial tissue. Structurally, the system includes a computer-controlled laser generating unit. Also, connected to the computer are a sensor for receiving the autofluorescent response, and an imaging unit for monitoring changes in the topography during a procedure. By monitoring both the autofluorescent response and changes in epithelial topography, the computer controls the laser unit. When there is no longer an autofluorescent response, the procedure has been completed and the system is shut down.

### FIELD OF THE INVENTION

The present invention pertains generally to systems and methods for removing corneal epithelium tissue. More particularly, the present invention pertains to the photoablation of corneal epithelium tissue using a laser beam. The present invention is particularly, but not exclusively, useful as a system and method for corneal epithelium removal which monitors fluorescence epithelial tissue to ensure that the correct amount of tissue is removed.

### BACKGROUND OF THE INVENTION

The corneal epithelium forms a protective tissue layer on the front surface of the cornea. Structurally, the tissue of the epithelium layer is relatively soft and it is in contact with the tear film of the eye. Upon removal from the eye, the epithelium can completely replace itself from limbal stem cells within a few days with little or no loss of clarity.

During some surgical procedures it is often desirable to remove the corneal epithelium or portions thereof. For example, the removal of the epithelium is a necessary step in several corneal procedures, including (but not limited to) corneal opacity or scar removal, photorefractive keratectomy for treatment of refractive errors (PRK) and the treatment of anterior basement membrane corneal dystrophy (ABMD).

Heretofore, several methods have been used to remove the corneal epithelium. In general, these methods rely on chemical and/or mechanical processes. For example, the application of ethyl alcohol is often used to loosen or sever the connections that join the epithelium to the underlying Bowman's membrane and corneal stroma. Alternatively, rotating brushes, surgical knives, and other instruments have been used to remove the epithelium using mechanical means. Usually this is done by hand, and under an operating microscope. These methods are typically performed under topical anesthesia.

The use of lasers to remove the corneal epithelium has several advantages over the chemical/mechanical techniques described above. These include improved patient comfort and decreased trauma to the underlying cornea. In addition, as compared with chemical/mechanical techniques, laser removal often results in decreased liberation of cellular contents and their associated inflammatory components. Also, laser removal provides a more exact matching of the desired and actual zone of epithelial removal. Further, laser removal typically results in a shorter time for re-epithelialization after surgery, and avoids risks associated with using toxic chemicals, such as ethyl alcohol, on the ocular surface.

On the other hand, there are certain challenges associated with the use of lasers to remove the corneal epithelium. In particular, it is sometimes difficult to accurately determine during a laser procedure, when the epithelium has been completely removed. Two factors contribute to this difficulty. For one, the thickness of the corneal epithelium varies over the corneal surface, and varies inconsistently from eye to eye. For another, the thickness of the epithelial layer is not consistent from patient to patient. Plus, the corneal epithelium is difficult to visualize, making determination of the endpoint of removal difficult to ascertain.

Because of the inherent epithelial thickness variations described above, a somewhat complex laser treatment is typically employed to remove the entire epithelial layer without disturbing the underlying tissue layers (i.e. Bowman's Membrane or the stroma). In most cases, substantial negative consequences arise if the entire epithelium layer is not removed or if the underlying tissue layers are disturbed. In particular, if too little epithelium is removed, residual epithelial tissue that is left behind can interfere with subsequent procedures. For example, if the goal is to remove all of the epithelium to create new epithelial attachments to Bowman's layer (such as with treatment of ABMD), residual epithelial tissue can cause a treatment failure. As another example, if the goal is to remove the epithelium as a component of laser refractive surgery in a PRK procedure, the residual epithelium can result in unpredictable and irregular ablation patterns, with adverse visual consequences. This is made worse by the irregular thickness of the epithelial layer, such that the subsequent refractive treatment may cause elevations and depressions in the cornea that may not be amenable to correction with current technology.

Adverse consequences can also occur when tissue is inadvertently removed beyond the epithelium (i.e. in Bowman's membrane or the stroma). For example, if the removal is performed to treat ABMD, then removing the underlying layer (called Bowman's layer) can interfere with the treatment success. On the other hand, if the removal is performed as a step in a laser refractive correction (such as PRK) then an incomplete removal of the epithelial tissue can alter the refractive correction in the underlying corneal tissue and impair the visual outcome, which may require additional treatment.

In light of the above it is an object of the present invention to provide a system and method for corneal epithelium removal which monitors epithelium tissue removal to ensure that the correct amount of epithelium tissue is removed. Another object of the present invention is to remove a corneal epithelium without leaving residual epithelial tissue that can interfere with subsequent procedures. Still another object of the present invention is to safely remove a corneal epithelium without disturbing underlying tissue layers such as Bowman's Membrane and the stroma. Yet another object of the present invention is to provide a system and method for removing a corneal epithelium that are easy to use and comparatively cost effective.

### SUMMARY OF THE INVENTION

In accordance with the present invention, a laser system for removing tissue of a corneal epithelium includes a laser unit for generating a surgical laser beam. Once generated, the laser beam is directed onto target tissue in the corneal epithelium to photoablate the target tissue. During the photoablation procedure, an autofluorescent response from tissue of the epithelium is monitored to determine whether residual epithelial tissue remains.

In one aspect of the present invention, the autofluorescent response is induced by the surgical laser beam. In another aspect, an external light source, such as a light emitting diode (LED), provides light having a wavelength suitable for creating the autofluorescent response from the corneal epithelium. For the present invention, monitoring of the autofluorescent response can be accomplished by visual observation of the eye, or a sensor can be employed. In some cases, a display for presenting the autofluorescent response as an image can be employed. In any event, the autofluorescent tissue response is monitored during the laser procedure and laser unit output is stopped where an autofluorescent response is not detected or observed.

When a sensor is used to monitor the autofluorescent response, the system can include a control unit which is operationally connected to the sensor and the laser source. With this arrangement, the control unit receives an input from the sensor and provides a control signal to the laser source. When the sensor input indicates that an autofluorescent response is detected, a control signal is transmitted to the laser source to continue photoablation. Where the sensor input indicates that an autofluorescent response is not detected, a control signal is transmitted to the laser source to discontinue photoablation.

In a particular laser procedure protocol, an ablation zone in the epithelium is first identified. Next, topographical contour features on an anterior epithelial surface within the ablation zone are determined. For example, the topographical contour features can be determined using an Optical Coherence Tomography (OCT) device. Then, based on the contour features of the epithelial surface, a predetermined pathway for movement of the laser beam's focal point through tissue of the epithelium is developed. Tissue is then photoablated along the predetermined pathway. In some cases, the protocol can also include the sequential identification of another ablation zone for subsequent conduct of the protocol. Further, during the implementation of a protocol, where no autofluorescent response has been detected by the sensor, the protocol can be modified to indicate such a non-response.

In one process, the predetermined pathway is layered over the ablation zone using a sequence of an n number of raster patterns. In this process, each raster pattern is positioned in a layer at a respective predetermined elevation eₙ from the interface between the epithelium and Bowman's membrane of the cornea. During the event, photoablation is performed in each respective raster pattern according to contour features of the epithelial surface. Upon completion of a raster pattern at a specified contour elevation, the laser beam's focal point is selectively advanced through a predetermined contour interval distance, Δe, toward the epithelium/Bowman interface for photoablation along a successive raster pattern at the next (i.e. lower) contour elevation.

In another process in accordance with the present invention, the predetermined laser point pathway is segmented with a plurality of contiguous segments. For this process, each segment includes an ablation area that is determined by topographical contour features on the epithelial surface in the ablation zone. In more geometric terms, for this process, each segment extends through the epithelium from the ablation area to the interface between the epithelium and Bowman's membrane of the cornea.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic of the combination of the interactive components for a system in accordance with the present invention;

FIG. 2 is a top plan view of a topographical contour map of features within an ablation zone on the epithelial surface of the cornea;

FIG. 3A is a cross-section view of the epithelium and Bowman's membrane as seen along the line 3-3 in FIG. 2 depicting a layered technique for performing a photoablation protocol in accordance with the present invention; and

FIG. 3B is a cross-section view of the epithelium and Bowman's membrane as seen along the line 3-3 in FIG. 2 depicting a segmented technique for performing a photoablation protocol in accordance with the present invention.

### DESCRIPTION OF THE PREFERRED EMBODIMENTS

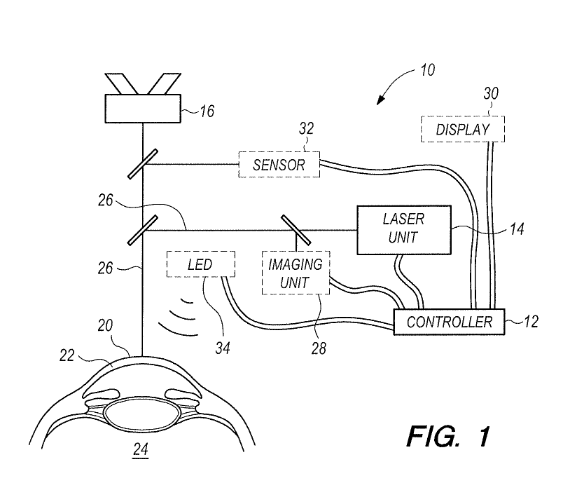

Referring initially to FIG. 1, a system in accordance with the present invention is shown and is generally designated 10. For a basic embodiment of the present invention, the system 10 includes a controller 12, and a laser unit 14 which is electronically connected with the controller 12. Additionally, the system 10 includes an eyepiece 16 which can be used by the system user (not shown) for viewing the epithelium 20 on the stroma 22 of an eye 24. In this combination, the controller 12 can be used to activate the laser unit 14 for the generation and control of a laser beam 26 as it is directed and focused onto the epithelium 20 of the eye 24. As intended for the present invention, control of the laser beam 26 by the controller 12 will result in the photoablation of epithelial tissue. For the basic embodiment, the user can view the photoablation process using the eyepiece 16.

For a more automated embodiment of the present invention, the system 10 can also include an imaging unit 28 which is electronically connected with the controller 12 for viewing the epithelium 20. More specifically, in this automated embodiment, a display 30 is also connected with the controller 12 for visually presenting images that are generated by the imaging unit 28.

An important aspect of the present invention is the capability of the system 10 to detect when a predetermined portion of the epithelium 20 has been completely removed without affecting other tissues of the eye 24. To do this, the present invention relies on an autofluorescent response that will result when the laser beam 26 interacts with tissue of the epithelium 20. When a sensor 32 is used for detecting the autofluorescent response, the response is transferred by the controller 12 for presentation on the display 30.

As disclosed above, the system 10 of the present invention is intended to detect an autofluorescent response that will result when tissue of the epithelium 20 is photoablated by the laser beam 26. For one embodiment of the present invention, the detection of an autofluorescent response is accomplished by direct visual observation, such as by a user (e.g. attending physician not shown) viewing the epithelium 20 through the eyepiece 16. In this case, the wavelength of the laser beam 26 must be capable of causing autofluorescent response, as well as performing the required photoablation. Accordingly, a laser beam 26 having a wavelength in the far violet or near ultraviolet wavelengths, will be required. For the other embodiment, the detection is accomplished by the sensor 32. In this case, it will be necessary to employ a Light Emitting Diode (LED) 34 that is capable of inducing the autofluorescent response. A Wood's lamp could be used.

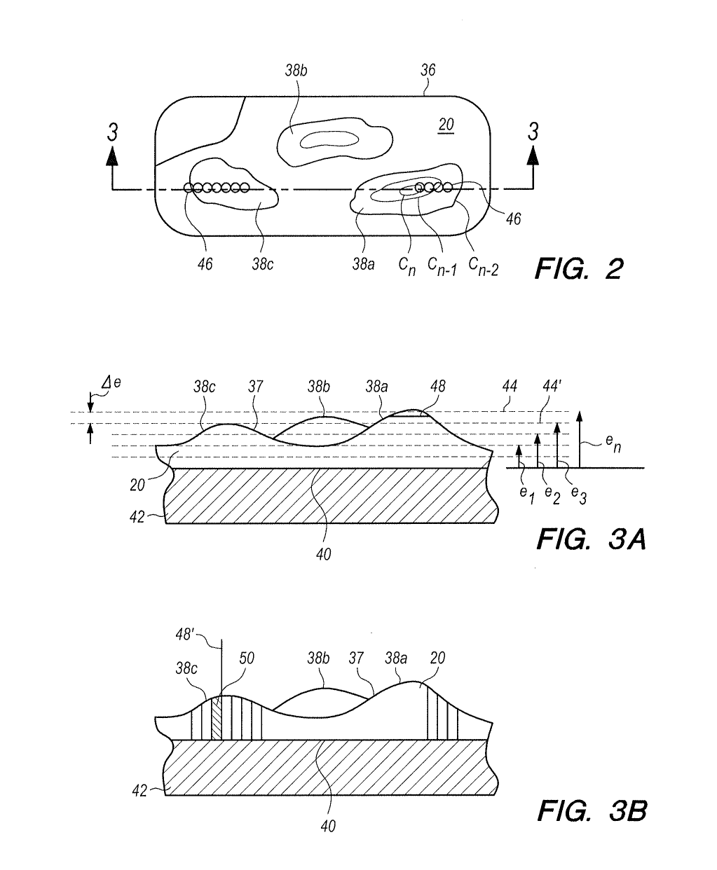

Referring now to FIG. 2, a zone 36 of the anterior surface 37 of the epithelium 20 is shown in a top plan view. As shown in FIG. 2, the zone 36 includes the exemplary topographical features 38a, 38b and 38c which are representative of typical irregularities on the anterior surface 37 of the epithelium 20.

With reference to FIG. 3A it will be appreciated that different elevations, eₙ, on the various topographical features 38 can all be referenced to the interface 40 that is located between the epithelium 20 and Bowman's membrane 42. As is well known in the pertinent art, a cross-section view of the zone 36 can be provided by the imaging unit 28 using well known imaging techniques, such as Optical Coherence Tomography (OCT).

By cross-referencing FIG. 2 with FIG. 3A for purposes of disclosure, it will be appreciated that a series of contour lines, cₙ, with each contour line cₙ having a same elevation eₙ in the zone 36, can be used to define the topographical features 38. Further, a contour interval, Δe, between adjacent contour lines (e.g. cₙ and cₙ₋₁) can be established. Importantly, Δe will depend on the extent to which tissue of the epithelium 20 is photoablated at a focal point of the laser beam 26. Thus, based on Δe, n will equal the number of horizontal photoablation layers 44, or vertical photoablation events 46, that must be performed to remove tissue of the epithelium 20 from the zone 36. In any event, once the contour interval, Δe, has been determined, the elevation, eₙ, of different contour lines, cₙ, can also be determined. Initially, of course, within the zone 36 the highest elevation for tissue in the topography of the epithelium 20 will be eₙ. For example, within this scheme, the contour line cₙ₋₂ will designate an elevation of eₙ₋₂ above the interface 40 for the third horizontal photoablation layer 44 or the third vertical photoablation event 46.

With the above in mind, and with reference to FIGS. 3A and 3B, it will be appreciated that an operation of the present invention can be performed essentially in either of two different ways. For one (see FIG. 3A), tissue of the epithelium 20 can be removed by photoablating tissue in a sequence of layers 44. In this case, the first layer 44 will be at the elevation eₙ above the interface 40. As envisioned for the present invention, for a removal of tissue by layers 44, the pathway 48 for the focal spot of laser beam 26 will be a horizontal straight line that, typically, will be part of a raster pattern. It is to be further appreciated that the pathway 48 will then continue on a subsequent raster pattern over the layer 44′ at the elevation eₙ₋₁, and so on. Another way for photoablating tissue in accordance with the present invention is by removing tissue of the epithelium 20 in vertical segments 50 (see FIG. 3B). In this case, the pathway 48′ for focal points of the laser beam 26 will be a straight, vertical line that is substantially normal to the interface 40. Further this pathway 48′ will extend from the elevation eₙ of the anterior surface 37 of the epithelium 20 for a segment 50 to the interface 40. Another segment 50 can then be identified, and the process repeated, as needed.

It is to be further appreciated that a combination of the horizontal and vertical photoablation procedures disclosed here can be used together if desired. Regardless how employed, the resultant autofluorescent response is monitored and, whenever there is no such response, the conclusion is that all tissue of the epithelium 20 that was above the interface 40 has been removed. Depending on the absence of an autofluorescent response, or an indication from the imaging unit 28 that epithelial tissue remains in the zone 36, an operation of the present invention is either stopped, continued as indicated, or it is moved to another zone 36 where epithelial tissue still remains.

While the particular System and Method for Removing Corneal Epithelium as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A laser system for removing tissue of a corneal epithelium from an eye which comprises:
a laser unit for generating and directing a surgical laser beam to a focal point to perform photoablation of target tissue in the corneal epithelium of the eye in accordance with a predetermined protocol;
a sensor for monitoring an autofluorescent response from tissue of the epithelium during conduct of the protocol; and
a control unit connected to the laser unit and to the sensor, wherein the control unit can be selectively activated to cease an operation of the laser unit, and to modify the protocol to indicate, where no autofluorescent response has been detected by the sensor.

2. The system as recited in claim 1 further comprising an external light source for providing light having a wavelength suitable for creating the autofluorescent response from the corneal epithelium.

3. The system as recited in claim 2 wherein the external light source is a Light Emitting Diode (LED) having a wavelength selected from the spectrum of ultraviolet (UV) and near UV wavelengths.

4. The system as recited in claim 2 further comprising a display for presenting the autofluorescent response as an image.

5. The system as recited in claim 1 wherein the autofluorescent response is induced by the surgical laser beam.

6. The system as recited in claim 1 wherein the protocol requires an identification of an ablation zone in the epithelium; a determination of topographical contour features on an anterior epithelial surface within the ablation zone; and instructions for installation of a predetermined pathway based on the contour features of the epithelial surface for movement of the laser beam's focal point along the pathway through tissue of the epithelium.

7. The system as recited in claim 6 wherein the determination requirement for the protocol is accomplished using an Optical Coherence Tomography (OCT) device.

8. The system as recited in claim 6 wherein the predetermined pathway is layered over the ablation zone using a sequence of an n number of raster patterns, wherein each raster pattern is positioned in a layer at a respective predetermined elevation eₙ from the interface between the epithelium and Bowman's membrane of the cornea, wherein photoablation is performed sequentially with a succession of selected raster patterns according to contour features of the epithelial surface, and further wherein upon completion of a raster pattern at a specified contour elevation, eₙ, the laser beam's focal point is selectively advanced through a predetermined contour interval distance, Δe, toward the epithelium/Bowman's interface for photoablation along a successive raster pattern.

9. The system as recited in claim 6 wherein the predetermined pathway is segmented with a plurality of contiguous segments, wherein each segment includes an ablation area determined by topographical contour features on the epithelial surface in the ablation zone, and each segment extends through the epithelium from the ablation area to the interface between the epithelium and Bowman's membrane of the cornea.

10. The system as recited in claim 6 wherein the protocol further requires a sequential identification of another ablation zone for subsequent conduct of the protocol.

11. A method for removing tissue of a corneal epithelium from an eye which comprises the steps of:
generating and directing a surgical laser beam to perform photoablation of target tissue in the corneal epithelium of the eye in accordance with a predetermined protocol;
monitoring an autofluorescent response from tissue of the epithelium during conduct of the protocol;
modifying the protocol to indicate where no autofluorescent response has been detected during the monitoring step;
continuing the generating and directing step when an autofluorescent response is detected; and
ceasing the generating and directing step where no autofluorescent response is detected.

12. The method as recited in claim 11 wherein the monitoring step is performed by visually observing the autofluorescent response.

13. The method as recited in claim 11 wherein the monitoring step is performed by a sensor.

14. The method as recited in claim 11 wherein step of generating and directing a surgical laser beam includes the step of focusing the laser beam to a focal point.

15. The method as recited in claim 14 wherein the predetermined protocol comprises the steps of identifying an ablation zone in the epithelium; determining topographical contour features on an anterior epithelial surface within the ablation zone; and establishing a predetermined pathway based on the contour features of the epithelial surface for movement of the laser beam's focal point along the pathway through tissue of the epithelium.

16. The method as recited in claim 15 wherein the determining step for the protocol is accomplished using an Optical Coherence Tomography (OCT) device.

17. The method as recited in claim 11 wherein the autofluorescent response from tissue of the epithelium is generated by light from a Light Emitting Diode (LED).

18. The method as recited in claim 11 wherein the autofluorescent response from tissue of the epithelium is induced by the surgical laser beam.

19. The method as recited in claim 11 wherein the continuing and ceasing steps are performed by a control unit connected to a laser unit and a sensor.

20. Non-transitory computer-readable medium having executable instructions stored thereon that direct a computer system to perform a process of removing tissue of a corneal epithelium from an eye that comprises generating and directing a surgical laser beam to perform photoablation of target tissue in the corneal epithelium of the eye in accordance with a predetermined protocol; monitoring an autofluorescent response from tissue of the epithelium during conduct of the protocol; modifying the protocol to indicate where there has been no autofluorescent response during monitoring; continuing to generate and direct the surgical laser beam onto the target tissue when an autofluorescent response is detected; and ceasing to generate and direct the surgical laser beam onto the target tissue where no autofluorescent response is detected.
